(19) United States
(12) Patent Application Publication
(10) Pub. No.: US 2014/0114231 A1
(43) Pub. Date: Apr. 24, 2014

(54) Formulations and Compositions of Hydrophobic Extracts of Skin Tree

(71) Applicants: Bertha Rostro, Houston, TX (US); Mehdie Kohanloo, Houston, TX (US); Dana Johnson, Houston, TX (US); Candace Johnson, Houston, TX (US)

(72) Inventors: Bertha Rostro, Houston, TX (US); Mehdie Kohanloo, Houston, TX (US); Dana Johnson, Houston, TX (US); Candace Johnson, Houston, TX (US)

(21) Appl. No.: 13/656,705

(22) Filed: Oct. 20, 2012

(51) Int. Cl. A61N 5/06; A61K 36/48

(52) U.S. Cl. CPC A61N 5/062; A61K 36/48
USPC 604/20; 424/757; 424/445; 44/307; 585/240

(57) ABSTRACT

This patent discloses formulations, compositions, and process method and device, of hydrophobic extracts and biomass of Skin Tree, also called Mimosa tenuiflora, also called Mimosa cabrera, also called Mimosa hostilis, also called Jurema, also called Tepezcohuite, and apparatus used for cell repair, and the regeneration and rejuvenation of glabrous skin and hair bearing skin. The formulations and compositions herein disclosed, alone and when used with apparatus described, i.e. in combination, will serve to modulate cell repair and block inflammatory processes, infection and disease, reversing skin aging, thereby restoring normal cell and skin conditions, coloration, homeostasis, youth, vitality, and firmness. Still other applications include biomass, biofuel processing methods, applications to biodiesel, gasoline, jet fuel, terpene fuel production, cellulose fuels, kerogens, crude oils, light sweet crude oil productions, refinery operations, viscosity modification, and refining, and deepwater oil exploration, crude oil desalting, fuels, biofuels, and fuel additives, refinery additives, or chemical modifiers, viscosity modifiers, and crude oil desalting.

This patent discloses formulations, compositions, and process method and device, of hydrophobic extracts and biomass of Skin Tree, Mimosa tenuiflora, Mimosa cabrera, Mimosa hostilis, Jurema, and Tepezcohuite, and apparatus used for the cell repair, regeneration and rejuvenation of glabrous skin and hair bearing skin. The formulations and compositions herein disclosed, alone and when used with apparatus described, refer to FIG. 1 LED Apparatus Horizontal View with legend showing: 1-Anode (−), 2-Cathode (+), 3-Encapsulation for LED Device, 4-Conductive Layer (+), and 5-Emission Layer (−); FIG. 2 LED Cross-Sectional View with legend showing: 3-Encapsulation for LED Device, 4-Conductive Layer (+), 5-Emission Layer (−), 6-LED Electronic Device Symbol with Cathode (+) and Anode (−), and 7-Vector Orientation Axes; FIG. 3 LED Apparatus Vertical View with legend showing: 1-Anode (−), 2-Cathode (+), 3-Encapsulation for LED Device, 4-Conductive Layer (+), and 5-Emission Layer (−); FIG. 4 LED Apparatus Back View with legend showing: 1-Anode (−), 2-Cathode (+); i.e. in combination, will induce an anti-inflammatory, anti-bacterial, anti-fungal, and anti-viral effect on cell lines such as dermis, reducing cutaneous inflammation and/or inflammatory responses, thereby removing erythema, necrotizing fasciitis, purpura fulminans, accelerating debridement, all of which serve to block the onset and proliferation of cell inflammatory processes which lead to trauma, wound formation, infection and disease, further reversing wrinkles, trauma, skin aging, and UV damage, thereby restoring normal skin coloration, youth, vitality, and firmness to skin. Formulations, compositions, and process method and device, of hydrophobic extracts of Skin Tree regulates IL-8 and its ability to serve in a pro-inflammatory role, reduces IL-1 inflammation, modulates IL-2 and IL-3 and IL-12, affects IL-4 allergic inflammation modulation, modulates IL-5 skin eosinophil recruited inflammation, regulates IL-6 and IL-7 for skin and dermal repair processes, modulates IL-9 and IL-10 irritation and/or shock and/or UV damage, affects IL-11 regulation and cytoprotection, affects IL-13 thereby regulating collagen homeostasis, affects IL-15 to minimize hypertrophic and/or normotrophic and/or pathologic scar formation, modulates IL-16 to decrease bulbous pemphigoid blistering through p38 MAP kinases, regulates IL-17 to reduce skin atopic lesions, modulates IL-18 to reduce skin inflammatory responses and disease, modulates IL-19 and IL-20 to reduce skin lesions, modulates IL-21 to offset epidermal hyperplasia, modulates IL-22 to restrict skin pro-inflammatory responses, modulates IL-23 to control and regulate skin pro-inflammatory responses and lesions, modulates and regulates IL-24 for wound repair, modulates and controls IL-25 which dictates inflammation, affects IL-26 modulation of cutaneous inflammation, affects IL-31 modulation for itch sensation such as in dermatitis, affects IL-32 modulation for skin inflammation, affects IL-33 which is active in skin inflammations and skin diseases in general, and which then act on MAP kinase signaling paths, and TGF-beta-1, serving to offset UV irradiation, osmotic stress, heat shock, and which activates epidermal growth factor receptors, such as interleukin receptors, tumor necrosis factor receptors, platelet derived growth factor receptors, and platelet activation factor receptor signaling pathways, and which affect matrix metalloproteinases, to prevent and stop collagen degradation, and promote collagen and elastin formation, healing, and restoration, to reduce collagen fiber misalignment which lead to hypertrophic and keloid type scars. Still other applications of extracts include biomass, biofuel processing methods, applications to biodiesel, gasoline, jet fuel, terpene fuel production, cellulose fuels, kerogens, crude oils, light sweet crude oil productions, refinery operations, viscosity modification, and refining, and deepwater oil exploration, crude oil desalting, fuels, biofuels, and fuel additives, refinery additives, or chemical modifiers, viscosity modifiers, and crude oil desalting.

### BRIEF DESCRPTION OF THE DRAWINGS

FIG. 1 LED Apparatus Horizontal View with legend showing 1-Anode (−), 2-Cathode (+), 3-Encapsulation for LED Device, 4-Conductive Layer (+), and 5-Emission Layer (−).

FIG. 2 LED Cross-Sectional View with legend showing 3-Encapsulation for LED Device, 4-Conductive Layer (+), 5-Emission Layer (−), 6-LED Electronic Device Symbol with Cathode (+) and Anode (−), and 7-Vector Orientation Axes.

FIG. 3 LED Apparatus Vertical View with legend showing 1-Anode (−), 2-Cathode (+), 3-Encapsulation for LED Device, 4-Conductive Layer (+), and 5-Emission Layer (−).

FIG. 4 LED Apparatus Back View with legend showing 1-Anode (−), 2-Cathode (+).

### BACKGROUND OF THE INVENTION

Aging, stretching forces, weight loss, pregnancy, hormonal changes, excessive sun exposure, excessive UV exposure, trauma, infections or disease, will cause damage to collagen and elastin fibers, leading to skin that exhibits fibrosis, striae atrophicae, striae gravidarum, dermatochalasis, cutis laxa, chalazoderma, dermatochalasia, dermatolysis, cicatrices, dermatomegaly, generalized elastolysis, generalized elastorrhexis, pachydermatocele, pemphigoid gestationis, polymorphic eruptions, venous ulcers, hyperkeratosis, rhytides, pruritic urticarial, glycational processes, hyperpigmentation, atrophic scarring, and generalized dermatitic conditions, which show excessive trauma, inflammation, stretching, dermal and epidermal tearing of fibers, and deficiency of elastin and elastic fibers on skin, and other vascular anomalies.

Current methods aimed at improving the appearance, and regeneration and rejuvenation of glabrous skin and hair bearing skin, or in repairing skin and dermal damage induced from infection from fungus, bacteria, virus, or parasites, or methods aimed at removing and repairing keratosis, hyperkeratosis, cicatrices, rhytides, atrophic scarring, hyperpigmentation, trauma, lesions, aging skin, cutis laxa, striae, hormonal changes, excessive sun exposure, excessive UV exposure, pemphigoid skin lesions, polymorphic eruptions, venous ulcers, pruritic urticaria, and generalized dermatitic conditions, entail the use of serums, gels, lotions, creams, aqueous solutions, polymeric solutions, emulsions, oils, topical acid treatments, chemical peels. Still other methods employ dermabrasion processes, laser resurfacing, targeted photodynamic therapies, plasma regeneration, photothermolysis, dermal heating, and combined nanoparticle and microparticle wound healing therapies. Yet others employ phage or bacteriophage therapies, skin grafting, and skin transplantation to remove erythema, necrotizing fasciitis, purpura fulminans, in order to accelerate debridement and restore skin healing. These methods can however, be costly, time consuming, can have a degree of, moderate, or short effect on skin rejuvenation and skin regeneration, and in some cases poses a future infection danger and promote scarring.

This patent discloses formulations, compositions, and process method and device, of hydrophobic extracts and biomass, emulsions, or alcohol solutions of Skin Tree, also called Mimosa tenuiflora, also called Mimosa cabrera, also called Mimosa hostilis, also called Jurema, also called Tepezcohuite, that can be combined with aqueous or non-aqueous compositions, essential oils, creams, lotions, gels, solvents, surfactants, polymers, and buffers to make medical, pharmaceutical, cosmetological compositions, and formulations that modulate cell repair, such that c-fos and c-jun and AP-1 transcription factors, p38, JNK, and ERK, and other MAP kinase pathways are affected to reverses the inflammation, established trauma, removing skin damage such as, fibrosis, striae atrophicae, striae gravidarum, dermatochalasis, cutis laxa, chalazoderma, dermatochalasia, dermatolysis, cicatrices, dermatomegaly, generalized elastolysis, generalized elastorrhexis, pachydermatocele, pemphigoid gestationis, polymorphic eruptions, venous ulcers, hyperkeratosis, rhytides, pruritic urticarial, glycational processes.

These compounds, when used alone, or in combination with device, are used to treat skin with trauma, inflammation, stretching, dermal and epidermal tearing of fibers, and deficiency of elastin and elastic fibers on skin, and other vascular anomalies, such that anti-inflammatory, anti-bacterial, anti-fungal, and anti-viral effects are noted, reducing cutaneous inflammation and inflammatory responses, thereby removing erythema, all of which serve to block the onset and proliferation of cutaneous inflammatory processes which lead to trauma, wound formation, infection and disease, further reversing wrinkles, trauma, skin aging, and UV damage, thereby restoring normal skin coloration, youth, vitality, and firmness to skin.

Still other uses of Skin Tree extracts also called Mimosa tenuiflora, also called Mimosa cabrera, also called Mimosa hostilis, also called Jurema, and also called Tepezcohuite, stemming from extractions of pulverized and grounded stem, bark, and leaf materials, lead to compositions useful in biomass, biofuel processing methods, biodiesel, and gasoline, and jet fuel, and terpene fuel production, and cellulose fuels, and kerogens, and crude oils, and light sweet crude oil productions, and refinery operations, and viscosity modification, and refining, and deepwater oil exploration, and crude oil desalting, and fuel, and biofuel, and fuel additives, and refinery additives, and chemical, viscosity modifiers, and crude oil desalting.

Herein is disclosed formulations and compositions, when used alone, and when used with apparatus described herein, i.e. in combination, will modulate dermal and skin agents such as interlukins (IL), as epidermal growth factor receptors, tumor necrosis factor receptor, platelet derived growth factor receptors, and platelet activation factor receptor signaling pathways, and affect matrix metalloproteinases, to prevent and stop collagen degradation, and promote collagen and elastin formation and restoration. This will affect certain MAP kinase pathways such as c-fos and c-jun and AP-1 transcription factors, p38 MAP kinase pathways which cause or lead to inflammation, trauma, aging, scarring, and skin damage.

It should be understood at the outset that although illustrative implementation of one or more embodiments are provided below, such disclosed formulations, compositions, systems, and methods may be implemented using any number of compositions and formulations, methods, embodiments, techniques, whether currently known or in existence. This disclosure should in no way be limited to the illustrated, provided, discussed implementations, drawings, and techniques, compositions, formulations provided and described herein, but may be modified within the scope of the claims along with their full scope of equivalents.

While embodiments of the disclosure have been shown and described below, modifications thereof, can be made by one skilled in the art, without departing from the spirit and teachings of the disclosure. The embodiments described herein are exemplary only, and are not intended to be limiting. Many variations and modifications of the disclosure described herein are possible and are within the scope of the disclosure.

When a numerical range with a lower limit and upper limit is disclosed, any number and any included range falling within the range is specifically disclosed. In particular, every range of values described herein is to be understood to set forth every number and range encompassed within the broader range of values. Use of the words “and/or”, “and”, “or”, “such that”, “such as”, “but not limited to”, “optionally” with respect to any element of a claim is intended to mean that the subject element is required, or alternatively, is not required. Both alternatives are intended to be within the scope of the claim. Use of broader terms such as comprises, includes, having, at least, should be understood to provide support for narrower terms such as consisting of, consisting essentially of, comprised substantially of, etc. Also the terms in the claims have their plain, ordinary meanings, unless otherwise explicitly and clearly defined by the patentee.

### EXAMPLE 1

Wound gel heating ointment that requires the use of at least 2% glycerin, or optionally at least 4% oxidized alginate, or optionally at least 3% gelatin, or combinations thereof, and at least 80% of hydrophobic extracts and biomass extract of Mimosa tenuiflora, also called Mimosa cabrera, also called Mimosa hostilis, also called Jurema, also called Tepezcohuite also called Skin Tree, and at least 11% water, together with a tissue scaffolding material, and dressing material composed of gauze like material of at least 1% gelatin, and at least 1% alginate, and at least 1% glycerin, that has been coated and immersed with a solution of at least 1% propylene glycol, at least 1% aloe vera leaf juice, at least 1% tocopheryl acetate, at least 1% PEG-75 lanolin, at least 1% citric acid, and 1% disodium phosphate.

### EXAMPLE 2

Wound healing aqueous solution that requires the use of at least 2% glycerin, 4% oxidized alginate, or optionally 3% gelatin, and a 80% of hydrophobic extracts and biomass extract of Mimosa tenuiflora, also called Mimosa cabrera, also called Mimosa hostilis, also called Jurema, and also called Tepezcohuite, also called Skin Tree, and at least 5.5% methanol, and at least 5.5% water, with added calcium of at least 0.01%, titanium of at least 0.01%, titanium oxide of at least 0.01%, phosphorous of at least 0.01%, potassium of at least 0.01%, sapphire crystal of at least 5%, pH of at least 6.5-10, copper of at least 0.01%, iron of at least 2%, iron oxide of at least 0.01%, magnesium of at least 1%, aluminum oxide of at least 15%, sodium of at least 2%, zinc oxide of at least 0.01%, at least 1% PEG 3350, and/or optionally at least 1% PEG 8000, and/or optionally at least 1% PEG 20000, at least 1% 3D silica alginate scaffolds with interconnective honeycomb microchannels, at least 0.01% strontium, and at least 1% of manganese, and at least 1% vanadium.

### EXAMPLE 3

Cream, lotion, gel, ointment, which is composed of at least 1% water, at least 0.1% fragrance, and at least 1% cetyl alcohol, and at least 1% glycerol stearate, and at least 2% extra virgin coconut oil, and at least 2% grape seed oil, and at least 2% jojoba oil, and at least 2% rose hip oil, and at least 0.1% castor oil, and at least 1% cocoa butter, and at least 1% shea butter, and at least 0.1% lanolin, and at least 0.1% cineole, and at least 0.1% cinnamaldehyde, and at least 0.1% linalool, and at least 3% tocopheryl acetate, and at least 2% almond oil, and at least 1% coumarin, and mixtures thereof, with at least 20% of hydrophobic extracts and biomass extract of Mimosa tenuiflora, also called Mimosa cabrera, also called Mimosa hostilis, also called Jurema, also called Skin Tree, and also called Tepezcohuite, where said ingredients and respective concentrations add to 100%.

### EXAMPLE 4

Wherein such grounded stem, bark, and leaf materials of Mimosa tenuiflora, also called Mimosa cabrera, also called Mimosa hostilis, also called Jurema, also called Skin Tree, and also called Tepezcohuite, stemming from extractions lead to biomass, biomass residue, cellulose compounds following processing such as, supercritical fluid extraction with solvents such as water, carbon dioxide and terpenes, water cavitation extraction, microwave cavitation extraction, steam extraction, turbine steam extraction, steam stripping, hydrothermal extraction, autoclave extraction, low temperature extractions, and sonication, carried out at pressures from 50 kPa to 100 MPa and temperatures from −25 to 800 C., such that resulting solution gives high yields of extracts such as terpenes and terpenoids that can be processed into biofuels or fuels, or used as additives in gasoline, fuel additives, jet fuels, biodiesel, alcohols, or for processing of kerogen compounds such that resulting solutions can be centrifuged and filtered, and solvents removed to leave fuels, additives, and respective species.

Accordingly, the scope of protection is not limited by the description set out herein, but is only limited by the claims which follow, that scope including all equivalents of the subject matter of the claims. Each and every claim is incorporated into the specification as an embodiment of the present disclosure. Thus the claims are a further description and are an addition to the embodiments of the present disclosure. The discussion of a reference herein is not an admission that it is prior art to the present disclosure, especially any reference that may have a publication date after the priority date of this application. The disclosures of all patents, patent applications, and publications cited herein are hereby incorporated by reference, to the extent that they provide exemplary, procedural, or other details supplementary to those set forth herein.

### DESCRIPTION OF PRIOR ART

Numerous methods are aimed at improving the appearance, and regeneration and rejuvenation of glabrous skin and hair bearing skin, or in repairing skin and/or dermal damage. Most include entail the use of serums, gels, lotions, creams, aqueous solutions, polymeric solutions, emulsions, oils, topical acid treatments, chemical peels, dermabrasion processes, laser resurfacing, targeted photodynamic therapies, plasma regeneration, photothermolysis, dermal heating, and/or combined nanoparticle and/or microparticle wound healing therapies, phage therapies, skin grafting, stem cell lines, skin transplantation. These methods can however, have a degree of, moderate, or short effect on skin rejuvenation and or skin regeneration, and in some cases pose a future infection danger and/or promote scarring. Moreover, other therapies may not have cell regeneration or rejuvenation properties. This patent discloses formulations and compositions of hydrophobic extracts, emulsions, or alcohol solutions, and biomass of Skin Tree, Mimosa tenuiflora, Mimosa cabrera, Mimosa hostilis, Jurema, and/or Tepezcohuite, where disclosed formulations and compositions, when used alone, and/or when used with apparatus described herein, i.e. in combination, will modulate cell repair mechanisms, or which can alternatively be used in oil-gas applications. A method to manufacture a therapeutic powder from the bark and outer layer of sap wood from the Mimosa Tenuiflora poir tree, has been disclosed in U.S. Pat. No. 4,883,663 wherein this method requires the chemical solvent and manual washing, roasting, and separation of curative active ingredients, followed by grinding of powder, sterilization, and use on burns, wounds, lesions, and/or other skin injuries. U.S. Pat. No. 5,122,374 discloses a method for producing an active ingredient isolated from the cortex of Mimosa tenuiflora using solvent extraction, where disclosed active ingredient shows epidermal regenerative properties which primarily are used for burn treatment, this disclosure used chloroform extraction, ethanol extraction, water extraction, concentration, drying, powder generation, wherein extraction is lixivation-percolation, maceration, continuous extraction to produce an extract that is only soluble in ethanol, DMSO, methanol, and acetone, but insoluble in water, chloroform, ethyl acetate, ethyl ether, propanol, hexane, CCl4, THF, dioxane, amyl alcohol, isopropyl alcohol, benzyl alcohol, and diethylamine. U.S. Pat. No. 5,885,564 discloses methods of use for compositions of certain nutrients, active, and protective substances, oxygen carriers, and process for preparation entailing use of phospholipids, fluorocarbons, phosphatidylcholine, plant substances such as bark of the Mexican skin tree Mimosa tenuiflora, and other plant materials, bacteria, yeasts, such that using ultrasonic and/or high pressure homogenization, together and/or in combination with solvents and disclosed formulation, are used as cosmetic or dermatological products. U.S. Pat. No. 6,342,208 discusses oil-in-water emulsions at pH 6 for application on skin surfaces whereby an oily and aqueous phase comprise a lipid of vegetable or animal origin that is at least 50% w/w of C₁₀-C₂₄ hydrocarbon carboxylic acid or mixtures thereof, which is used to treat human skin, against parasites, and for conferring sun protection, wherein disclosed lipids are mixed with extracts from the group of which Mimosa tenuiflora bark can be used upon stabilization with surfactant/emulsifier. U.S. Pat. No. 6,416,769 describes a cosmetic composition(s) for topical sue that comprises an exfoliating enzyme for use in the removal of at least one portion of the dead skin cells from the outer layer of skin, in conjunction with one or more botanicals, where the delivery of such to subsurface layers and cells thereof of skin is enhanced by disclosed exfoliating enzyme which is papain with select additives of which a Mimosa tenuiflora extract is mentioned in combination with an array of other ingredients with major ones being Echinacea and hydrocotyl extracts. U.S. Pat. No. 6,426,080 describes cosmetic preparations of active substances that protect the skin against free radical aggression, both alone or in combination with other active ingredients, consisting of Quebraco blanco bark extract, silkworm extract, vitamin mixtures, amino acids, non-ionic, cationic, anionic hydrogels, phospholipids, water, vitamin derivatives and plant extracts of acerola, sea weed, citrus, bitter orange, cherry, papaya, tea, coffee beans, and Mimosa tenuiflora, and angelica. Still another similar application, that being U.S. Pat. No. 6,793,932 discusses a veterinary composition comprising at least one keratolytic and cerumenilytic cleaning agent, with at least one bactericide agent, at least one yeast control agent, and at least one anti-irritant and anti-pruriginous agent, which is useful for preventing otitis in dogs and cats, wherein composition is composed of lactic acid, salicylic acid, Cetraria islandica extracts, Cucumis sativus vegetable extract which further comprises vegetable extracts of Mimosa tenuiflora, and Camomilla recutica. U.S. Pat. No. 6,989,150 discusses inventive cosmetic preparations of active substances, which in combination with other active substances, protects the skin against free radical aggression, whereby disclosed composition consists of Quebracho blanco, silkworm extract, cecropine, amino acids, vitamin mixtures, non-ionic, cationic, anionic hydrogels, phospholipids, yeast disintegration products, cyclodextrins, with plant extracts of acerola, sea weed, citrus, bitter orange, cherry, papaya, tea, coffee beans, extracts of the bark Mimosa tenuiflora, and angelica, among other extracts and compositions. U.S. Pat. No. 7,241,456 discloses a topical delivery of bioactive agents including anhydrous formulations for percutaneous absorption of high concentrations that are non-irritating, wherein, niacin, aspirin, ibuprofen, acetaminophen, cox-2 inhibitors, esters, amides, exthoxylated fats, mineral oil, petrolatum, vegetable oils, animal fats, triglycerides, wherein a biological additive of Mimosa tenuiflora extract is included. We claim formulations, compositions, and process method and device, that are at least 1 to 100% of hydrophobic extracts and/or biomass of Skin Tree, Mimosa tenuiflora, Mimosa cabrera, Mimosa hostilis, Jurema, and/or Tepezcohuite, stemming from extractions of pulverized and grounded stem, bark, and leaf materials, and apparatus used for the restoration, repair, and regeneration of cells, and rejuvenation of such cells, meaning that collagen degradation is prevented and stopped, and collagen and elastin formation is promoted, due to healing, and restoration, with reduction and/or prevention of collagen fiber misalignment and removal of hypertrophic and keloid type scarring, while other formulations and compositions are used in oil-gas applications as described below.

### SUMMARY OF THE INVENTION

We claim a formulations, compositions, and process method and device, that are hydrophobic extracts and biomass that are at least 1 to 100% of Skin Tree also called Mimosa tenuiflora, also called Mimosa cabrera, also called Mimosa hostilis, also called Jurema, and also called Tepezcohuite, stemming from compositions from pulverized and grounded stem, bark, biomass, and leaf materials; and apparatus used for the repair of cells, regeneration of human cell lines, and rejuvenation of such cells that prevent and stop collagen degradation, and promote collagen and elastin formation, healing, and restoration, while reducing and preventing collagen fiber misalignment and removing hypertrophic and keloid type scarring, other formulations promote cell growth and repair, while other formulations and compositions find use in various oil-gas applications.

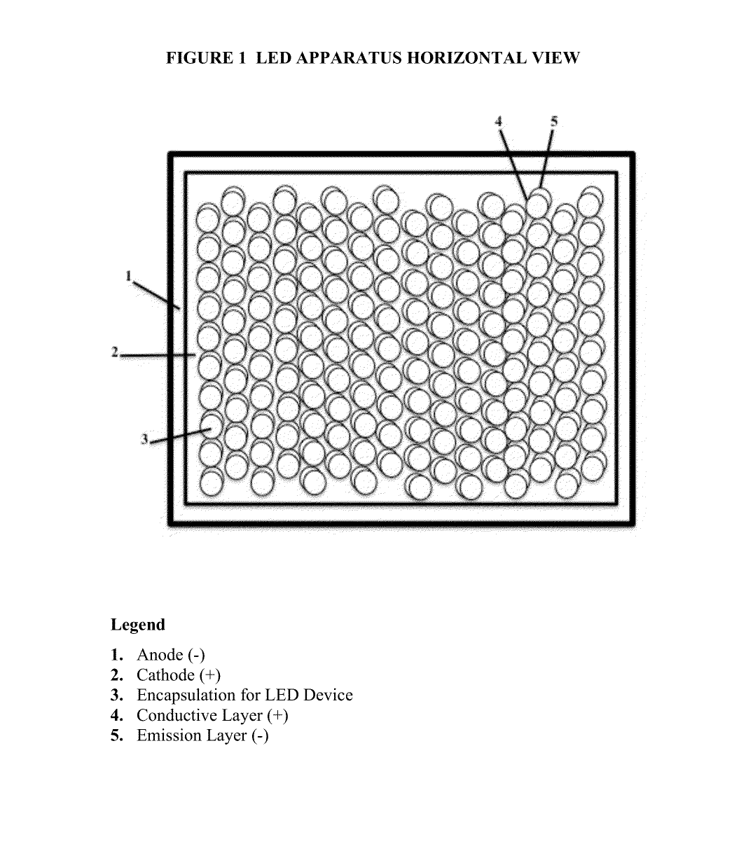

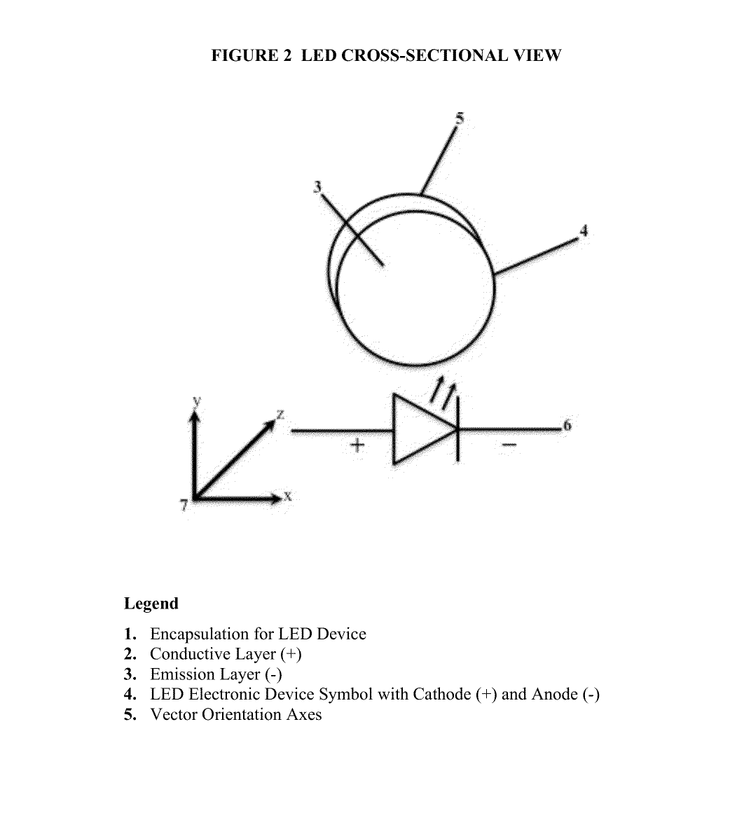

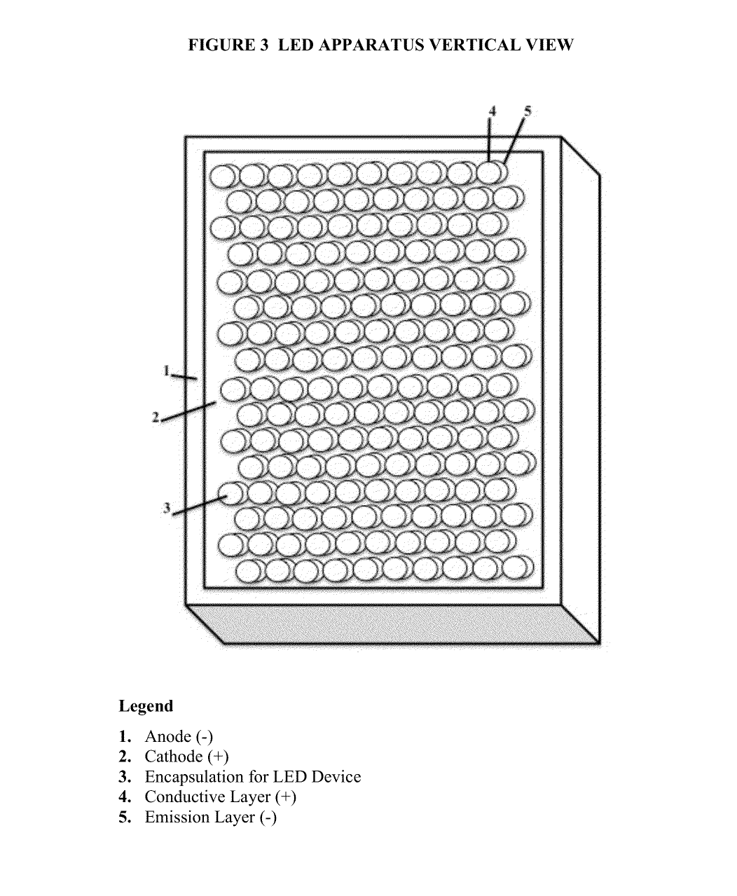

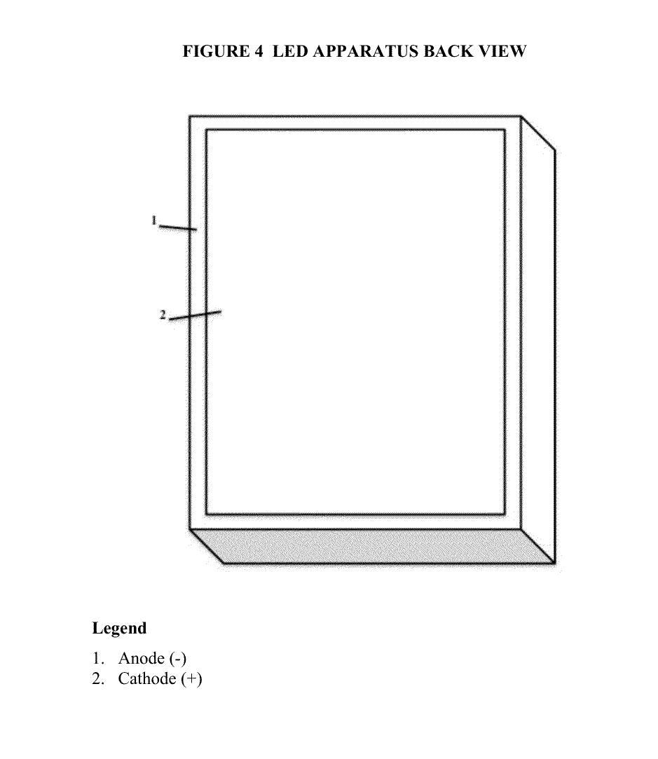

1. We claim a process method and device, as shown in FIG. 1 LED Apparatus Horizontal View with legend showing: 1-Anode (−), 2-Cathode (+), 3-Encapsulation for LED Device, 4-Conductive Layer (+), and 5-Emission Layer (−); FIG. 2 LED Cross-Sectional View with legend showing: 3-Encapsulation for LED Device, 4-Conductive Layer (+), 5-Emission Layer (−), 6-LED Electronic Device Symbol with Cathode (+) and Anode (−), and 7-Vector Orientation Axes; FIG. 3 LED Apparatus Vertical View with legend showing: 1-Anode (−), 2-Cathode (+), 3-Encapsulation for LED Device, 4-Conductive Layer (+), and 5-Emission Layer (−); FIG. 4 LED Apparatus Back View with legend showing: 1-Anode (−), 2-Cathode (+); comprising steps that use hydrophobic extracts that are at least 1 to 100% and hydrophobic extracts stemming from extractions of pulverized and grounded stem, bark, and leaf materials of Mimosa tenuiflora, also known as Skin Tree also known as Mimosa cabrera, also known as Mimosa hostilis, also known as Jurema, or also known as Tepezcohuite, and apparatus used for cell repair, the regeneration of human cell lines, and rejuvenation of glabrous skin and hair bearing skin, wherein active ingredients alone and when combined with apparatus, affect human keratinocytes and dermal fibroblast proliferation and repair activity, yielding epidermal rejuvenation and regeneration, serving to maintain and restore skin homeostasis which favors collagenogenetic induction, to prevent and stop collagen degradation, and promote collagen and elastin formation, healing, and restoration, removing hypertrophic and keloid type scarring while reducing and helping to prevent collagen fiber misalignment.

2. The method of claim 1 where the extracts from plant are obtained by taking pulverized grinded powder from stems, bark, and leaves that are filtered through 50 micron mesh, and then subjected to at least one process such as supercritical fluid extraction with supercritical water, or supercritical solvents such as carbon dioxide or terpenes, or cavitation such as water cavitation extraction, microwave cavitation extraction, steam extraction, steam stripping, hydrothermal extraction, autoclave extraction, low temperature extractions, or sonication, at pressures from 50 kPa to 100 MPa and 20 bars to 400 bars, and temperatures from at least −25 to 800 C or 250 K to 800 K, producing an extracted solution that is then centrifuged and filtered, and solvents removed, and concentrated, for example by rotavap evaporation, so that water content is removed either entirely or to some degree.

3. The method of claim 1 where extracted compounds from said Mimosa species leaf, stem, and bark includes biomass, biomass residue, plant based alcohol solutions, hydrophobic solutions, hydrophobic extracts, hydrophobic residues, aqueous solutions, tannins, sap, kerogens, cellulose, terpenoids, triterpenes, terpenes, saponins, triterpene saponins, lactones, ketones, galactans, condensed tannins, flavonoids, flavone aglycone, alkaloids, lipids, phytosterols, glucosides, xylose, lupeol, methoxychalcones, and kukulkanins, of these polyflavonoid tannins, tenuiflorin, capillarisin, catechol-type tannins, and non-hydrolyzable tannins, flavolans, of proanthocyanidins, prodelphinidins, leuco-fisetinidin, leucoanthocyanidin, fisetinidin, profisetnidins, proguibourtinidins, robinetidin, guibourtinidin, Quebracho tannins, prorobinetinidins, prorobinetidins, gallecatechins, to produce a brownish solution that is hydrophobic and amphiphilic.

4. The extract of claim 3 which is formulated for used in cosmetics, ointments, suspensions, gels, creams, lotions, soaps, aerosols, compresses, bandages, wound dressings, heat activated compresses, sauna suits, or body wrapping dressings to promote skin rebuilding following trauma, reducing hyperpigmentation, atrophic scarring, and generalized dermatitic conditions which show excessive trauma, inflammation, stretching, dermal and epidermal tearing of fibers, or deficiency of elastin and elastic fibers on skin, or other vascular anomalies, where formulations and compositions alone, or when used with apparatus described herein, i.e. in combination, will also induce an anti-inflammatory, anti-bacterial, anti-fungal, and anti-viral effect on dermis, reducing cutaneous inflammation or inflammatory response, thereby removing erythema, necrotizing fasciitis, purpura fulminans, accelerating debridement, all of which serve to block the onset and proliferation of cutaneous inflammatory processes which lead to trauma, wound formation, infection and disease, further inhibiting collagenase activity in skin and reversing wrinkles, trauma, skin aging, and UV damage, thereby helping to restore normal skin coloration, youth, vitality, and firmness to skin.

5. The extract of claim 4, wherein said formulation is used in body wraps and mud wraps with at least 58% methanol emulsion, which when combined with at least 7% of clay with at least a mineral content of at least 44.5% silica, silicon oxide at least 5%, bentonite, calcium of at least 0.01%, titanium of at least 0.01%, titanium oxide of at least 0.01%, phosphorous of at least 0.01%, potassium of at least 0.01%, sapphire crystal of at least 5%, moisture of at least 5%, pH of at least 6.5-10, copper of at least 0.01%, iron of at least 2%, iron oxide of at least 0.01%, magnesium of at least 1%, aluminum oxide of at least 15%, sodium of at least 2%, zinc oxide of at least 0.01%, at least 7% of sea salt, at least 7% mud volcano, at least 7% algal and marine phytoplankton powder, at least 7% coffee powder, at least 7% green tea with at least 85% catechin content, such that said formulation when combined device, and with LED therapy, photodynamic therapy, and laser therapies, will promote skin rejuvenation and skin regeneration.

6. The extract of claim 4, wherein, tissue scaffolds for skin repair, wound healing, and dermal reconstruction arise from at least 1% PEG 3350, and at least 1% PEG 8000, and at least 1% PEG 20000 coating a supporting 3D silica alginate scaffolds with interconnective honeycomb microchannels or nanochannels, with at least 0.01% strontium, at least 1% calcium, with at least 0.01% magnesium, with at least 0.01% zinc, with at least 0.01% copper (II) ions, with at least 0.1% iron (III) ions, with at least 0.1% manganese which enhance integrin expression and keratinocyte migration, with at least 0.1% vanadium, such that said formulation affects, modulates, and reduces inflammation, affects skin and dermal repair processes, decreases irritation and UV damage, affects cytoprotection, thereby regulating collagen homeostasis, minimizes hypertrophic and normotrophic and pathologic scar formation, reduces skin atopic lesions, reduces skin inflammatory responses and disease, affects wound repair, dermatitis, serving to offset UV irradiation skin damage, to prevent and stop collagen degradation, and promote collagen and elastin formation, healing, and restoration, to reduce collagen fiber misalignment which lead to hypertrophic and keloid type scars.

7. The extract of claim 4, where a gel ointment that requires the use of at least 80% of hydrophobic extracts and biomass of Skin Tree also known as Mimosa tenuiflora, Mimosa cabrera, Mimosa hostilis, Jurema, and Tepezcohuite, 11% water, and at least 1% PEG 3350, and at least 1% PEG 8000, and at least 1% PEG 20000 coating, and supporting, together with a tissue scaffolding material, and dressing material composed of gauze like material of at least 1% gelatin, and at least 1% alginate, and at least 1% glycerin, that has been coated and immersed with a solution of at least 1% propylene glycol, at least 1% aloe vera leaf juice, at least 1% tocopheryl acetate, at least 1% PEG-75 lanolin, at least 1% citric acid, and 1% disodium phosphate, and 1% oxidized alginate, or combinations thereof.

8. The extract of claim 4 where a cream, ointment, lotion, or gel emulsion that requires the use of at least 5% of hydrophobic and biomass extracts and biomass of Skin Tree also known as Mimosa tenuiflora, also known as Mimosa cabrera, also known as Mimosa hostilis, also known as Jurema, also known as Skin Tree and also known as Tepezcohuite, and at least 1% tocopheryl acetate, and at least 1% water, at least 0.01% fragrance, and at least 0.1% cetyl alcohol, and at least 0.1% glycerol stearate, and at least 1% extra virgin coconut oil, and at least 1% jojoba oil, and at least 1% rose hip oil, and at least 1% castor oil, and at least 1% cocoa butter, and at least 1% shea butter, and at least 1% lanolin, and at least 0.1% cineole, and at least 0.1% cinnamaldehyde, and at least 0.1% linalool, and at least 0.01% coumarin, and at least 1% grapeseed oil, and at least 1% almond oil.

9. The extract of claim 4 that generates an astringent for skin or dermal, or cell line, and as aerosol based compositions that affect cell viability, and serves as antiseptic with antioxidants, and whereby at least 5% natural grain SD-alcohol 40, and at least 2% methanol, and at least 5% aloe leaf juice, and at least 1% pentalyne glycol, and at least 1% polysorbate 20, and at least 1% nymphaea alba root extract, and at least 1% polygala senega root extract, and at least 1% berberis vulgaris extract, and at least 1% algae extract, and at least 1% caffeine extract, and at least 1% dipotassium glycyrrhizate, and at least 1% trehalose, and at least 1% sodium hyaluronate, and at least 1% sodium pca, and at least 1% sucralose, and at least 1% urea, and at least 1% jojoba wax PEG 120 esters, and at least 1% glycerin, and at least 1% butylene glycol, and at least PPG-5-Ceteth-20, and at least 1% sodium citrate, and at least 1% polyquaternium-51, and at least 1% menthol, and at least 1% citric acid, and at least 1% disodium EDTA, and at least 1% chlorphenesin, and at least 1% phenoxyehtanol, and at least 1% with hazel extract, and at least 2% glycerin, and at least 1% fragrance, and at least 1% citric acid extract, and at least 1% grapefruit seed extract, and at least 1% chamomile extract, and at least 1% orange peel extract, and at least 1% blackberry extract, and at least 1% green tea extracts, and at least 1% cocoa extracts, and at least 1% lavender, and at least 1% mint leaf extract, and at least 1% rose hip seed extracts, and at least 1% coffee bean extracts, that is used to treat damaged, burned, scalded, acned, and traumatized skin.

10. The apparatus/device of claim 1, as shown in FIG. 1 LED Apparatus Horizontal View with legend showing: 1-Anode (−), 2-Cathode (+), 3-Encapsulation for LED Device, 4-Conductive Layer (+), and 5-Emission Layer (−); FIG. 2 LED Cross-Sectional View with legend showing: 3-Encapsulation for LED Device, 4-Conductive Layer (+), 5-Emission Layer (−), 6-LED Electronic Device Symbol with Cathode (+) and Anode (−), and 7-Vector Orientation Axes; FIG. 3 LED Apparatus Vertical View with legend showing: 1-Anode (−), 2-Cathode (+), 3-Encapsulation for LED Device, 4-Conductive Layer (+), and 5-Emission Layer (−); FIG. 4 LED Apparatus Back View with legend showing: 1-Anode (−), 2-Cathode (+); consisting of a photomodulation process that modifies skin and dermal cell activity using light without the need for a thermal effect, this being a non-thermal low dose flexible light emitting diode array, such as polychromatic light, where said light diodes emit photons at wavelengths ranging from 400 nm to 1500 nm light non-thermal full face LED array with 0.1 mW to 500 mW output power delivering at least 0.01 J/cm(2) to 100 J/cm(2) with specific pulsing sequence with select blue light, and purple light, and red light, and orange light, and yellow light, and green light, and combinations thereof, which has been transfer printed or transferred by non-conventional lithographies such as tape lithography methods, onto substrate, such as skin, dermis, human cell material, light and flexible substrates, composed of materials such as latex, and paper, and polymer, and glass, and gelatin, and cellulose, and metal, or composite, and circuit board, and leather, and films, and fibers, and ceramics, and clothing, and dressing, and gauze, and wrapping material, and wearable device, and implantable device, and tissue scaffold material, or organ material, or combinations thereof, such that optimum LED-LED spacing has the optimum packaging density, and uniform near- and far-field irradiance, and is at least 0.5 mm by 0.5 mm by 0.1 mm in measurement, and spaced at least 1 mm apart center-to-center, enabling a high signal-to-noise ratio, and where triboluminescence of peeling said materials produces and generates soft-xrays which further remove damaged cells, and work to generate soft-tissue imaging modalities.

11. The extract of claim 4, wherein, formulations and compositions, when used alone, and when used with apparatus described herein, i.e. in combination, will modulate cell regeneration, and growth factor receptors, and associated receptor signaling pathways, in respective organs and tissues such that organ regeneration occurs in tissue scaffolds and matrices, and affect matrix metalloproteinases, to prevent and stop collagen degradation, and promote collagen and elastin formation and restoration of cells in specific organs and associated tissues, such that labile, quiescent, non-dividing permanent tissues can be regenerated and restored to homeostasis in cells ranging from blood cell lineages, chondrocytes, osteoblasts, adipocytes, myoblasts, endothelial precursor cells, hepatocytes, neuronal cells, myocytes, satellite cells, keratinocytes, fibroblasts, kidney cells, bladder cells, uroepithelial cells, epithelial cells, endothelial cells, motoneuron cell lines, mesenchymal cells, macrophages, smooth muscle cells, fibronectin, such that matrix deposition and angiogenesis processes are modulated, enacted, and affected, such that MAP kinase pathways such as c-fos and c-jun and AP-1 transcription factors, and p38, JNK, ERK, and MAP kinase pathways are affected, to reduce and remove inflammation, trauma, aging, scarring, and skin damage.

12. The extract of claim 4, where a gel ointment that requires the use of at least 80% of hydrophobic extracts and/or biomass of Mimosa tenuiflora, also known as Mimosa cabrera, also known as Mimosa hostilis, also known as Jurema, also known as Skin Tree, and also known as Tepezcohuite, and at least 11% water, and at least 1% of 0.1 mM and higher of HEPES, and at least 1% of 0.1 mM and higher of TWEEN, and at least 1% of 0.1 mM and higher of citric acid, and at least 1% of 0.1 mM and higher of mM MES, and at least 1% of 0.1 mM and higher of MOPS, and at least 1% of 0.1 mM and higher of ascorbic acid buffer, and at least 1% of 0.1 mM and higher of CHOPS; and at least 1% PEG 3350, and at least 1% PEG 8000, and at least 1% PEG 20000, and at least 1% PEG 5000, and at least 1% PEG 5500, and at least 1% PEG 6000 coating, and supporting, together with a tissue scaffolding material, and dressing material composed of gauze like material of at least 1% hydrolyzed and unhydrolyzed gelatin, and at least 1% alginate, and at least 1% glycerin, that has been coated and immersed with a solution of at least 1% propylene glycol, at least 1% aloe vera leaf juice, at least 1% tocopheryl acetate, at least 1% PEG-75 lanolin, at least 1% citric acid, and 1% disodium phosphate, and 1% oxidized alginate; and at least 0.1% of barium, and at least 0.1% of calcium, and at least 0.1% magnesium, and at least 0.1% vanadium, and at least 0.1% strontium, and at least 0.1% zinc, and at least 0.1% silicates, and at least 0.1% barium-calcium-magnesium-strontium-zinc oxide-silicate, and at least 0.1% copper, and at least 0.1% chromium, and at least 0.1% cobalt, and at least 0.1% selenium, and at least 0.1% iron, and at least 0.1% manganese, and at least 0.1% molybdenum, and at least 1% chondroitin sulfate, and at least 1% glycine, and at least 1% proline, and at least 1% arginine, and at least 1% lysine, and at least 1% hydroxyproline, and at least 1% hyrdoxylisine, and at least 1% cortisol, and at least 1% cholecalciferol, and at least 1% ergocalciferol, and at least 1% Vitamin D, and at least 1% or higher of calcium phosphate; whereby such components will modulate cell regeneration, and growth factor receptors, and associated receptor signaling pathways, in respective organs and tissues such that organ regeneration occurs in tissue scaffolds and matrices, and affect matrix metalloproteinases, to prevent collagen degradation, and promote collagen and elastin formation and restoration of cells in specific organs and associated tissues, such that labile, quiescent, non-dividing permanent tissues can be regenerated and restored to homeostasis in cells ranging from blood cell lineages, chondrocytes, osteoblasts, adipocytes, myoblasts such that bone secretions of osteoid and osteocalcinin and collagen integrins are activated to regenerate bone.

13. The extract of claim 4, that yields biomass and is used in biomass processing, and yields biofuel agents, and biofuels such as biodiesel, gasoline, jet fuel, terpene fuel production, cellulose fuels, kerogen, crude oil, light sweet crude oil products, and yields refining chemicals that can be used for viscosity modification, and deepwater oil exploration, and crude oil desalting.

14. The material of claim 1, which are used for biomass, fuels, biofuels, terpenoids, alcohols, hydrogen, kerogen, biodiesel, high octane fuel, gasoline, jet fuel, terpene fuel production, aviation fuels, cellulose fuels, crude oil, light sweet crude oil production, refinery additives or chemicals, viscosity modifiers, and for implementation in deepwater oil exploration, and crude oil desalting, and other fuel additives such as alkadienes, alkatrienes.
